(19) United States
(12) Patent Application Publication
(10) Pub. No.: US 2024/0245737 A1
(43) Pub. Date: Jul. 25, 2024

(54) ISOLATED MICROORGANISM LACTIPLANTIBACILLUS PLANTARUM SNI3 STRAIN, FORMULATION BASED ON SNI3 STRAIN, ITS APPLICATION ON MALE VERTEBRATES, BREEDING MALES AND HUMAN MALES TO FAVORABLY CHANGE THEIR BEHAVIOR, TO DECREASE BODY FAT CONTENT AND INCREASE SPERM NUMBER AND TESTOSTERONE LEVEL AS WELL AS TO MANUFACTURE SNI3 BASED PREPARATIONS: POWDER, SOLUTION, SYRUP, MILK PRODUCT, CAPSULE, TABLET OR CHEWABLE TABLET PRODUCTS

(71) Applicants: Szilamér Imre FERENCZI, Kartal (HU); Krisztina KOVÁCS, Budapest (HU); INSTITUTE OF EXPERIMENTAL MEDICINE, Budapest (HU); ZYMBIOSYS RESEARCH AND ÉS DEVELOPMENT LTD;, Gödöllö (HU)

(72) Inventors: Szilamér Imre FERENCZI, Kartal (HU); Krisztina KOVÁCS, Budapest (HU); József KUKOLYA, Gödöllo (HU); Ildikó BATÁNÉ VIDÁCS, Budapest (HU); István NAGY, Szentendre (HU)

(21) Appl. No.: 18/566,643

(22) PCT Filed: Jun. 4, 2022
(86) PCT No.: PCT/HU2022/050050

§ 371 Date: Dec. 2, 2023

(30) Foreign Application Priority Data
Jun. 4, 2021 (HU) P2100212

(51) Int. Cl. A61K 35/747; A61P 3/02; A61P 15/10; C12N 1/20; C12R 1/25

(52) U.S. Cl. CPC A61K 35/747; A61P 3/02; A61P 15/10; C12N 1/205; C12R 2001/25

(57) ABSTRACT

The present invention provides our new isolate Lactiplantibacillus plantarum SNI3 bacterial strain, deposited as NCAIM (P) B 001482, formulation based on SNI3 strain, application of formulation on male vertebrates, breeding male and human males to favorably change their behavior and lean body mass, to decrease body fat content and increase their reproductive fitness, sperm number and testosterone level.

### SUBJECT OF THE INVENTION

Our invention is based on our isolate Lactiplantibacillus plantarum SNI3 bacterial strain, deposited as NCAIM (P) B 001482, formulation based on SNI3 strain, application of formulation on male vertebrates, breeding male and human males to favorably change their behavior and lean body mass, to decrease body fat content and increase their reproductive fitness, sperm number and testosterone level.

The microbial strain and the preparation thereof increases sperm count and serum testosterone level, while decreases characteristics of aggressive behavior and anxiety.

Feed and food supplements, containing Lactiplantibacillus plantarum SNI3 bacterial composition have synergistic effect on the above-mentioned body compositional-, reproductive physiological- and behavioral parameters.

Lactiplantibacillus plantarum SNI3 bacteria, produced as described, and heat or ultrasound pretreated if desired, will be transformed into food and feed supplements by adding carriers, excipients, which are commonly used in pharmaceutical technology and/or by adding prebiotic materials.

The main fields of application of the invention: to reduce reproductive problems of male vertebrates, breeding animals and males and to maintain healthy fertility. To increase/restore normal sperm number and sperm quality, which have been deteriorated by ageing, different environmental toxicants, poor and/or inadequate nutrition, unhealthy diets and excessive stress. Restoration of testosterone levels in andropause. Influencing aggressive behavior of male breeding animals in a calm, peaceful anxiety-free direction. Reduction of obesity, increase of muscle mass in humans and vertebrates.

The scope of the present invention also includes the use of the composition:
to develop ideal body composition via increasing lean body mass and decrease of body fat;
to maintain healthy reproductive status in male animals and human
to decrease male fertility problems
to increase/restore normal sperm number and sperm quality, which have been deteriorated by ageing, different environmental toxicants, poor and/or inadequate nutrition, unhealthy diets and excessive stress
to restore decreased testosterone levels in andropause□ to decrease anxiety and aggression in males.

### Definitions of Terms and Definitions Used in the Description

1. Bacterial strain: a genetic variant or subtype of a bacterium. e.g. a Lactobacillus strain is a lactic acid bacterium with an exclusive genetic information specific to it.

Lactiplantibacillus plantarum SNI3 strain has been isolated in 1993 from the feces of a breeder swine in Sajobábony (Hungary). The strain has been deposited in National Collection of Agricultural and Industrial Microorganisms, Institute of Food Science and Technology, Hungarian University of Agriculture and Life Sciences (Lactiplantibacillus plantarum SNI3 NCAIM (P) B 001482).
2. CFU: Colony-forming unit. Number of a given microorganism, which are able to produce viable colonies. The concentration of the microorganism is given in CFU/ml for liquid and CFU/g for solid products.
3. Testosterone: The most important male sex hormone produced by the testis, which has a prominent role in the development of male secondary sex characteristics (male-specific bone and skeletal muscle, body hair, behavior).
4. Grouping of the species mentioned in the invention:
   4.1. Human: From a taxonomic point of view, the human genus belongs to the kingdom of animals, to the order of the primates, to the family of hominidae (Hominidae). Humans living today belong to the species Homo sapiens (“intelligent man”). According to the biological sex, humans are woman or man.
   4.2. Farm animals: the breeding of which is closely linked to agriculture. During domestication people have introduced some herds of the wild animal species into artificially controlled breeding, creating artificial breeds through artificial selection.
      4.2.1. Livestock: animals, bred for economic gain are referred to as farm animals: vertebrates, of which, but not limited to: pigs, cattle, sheep, goats, rabbits, horses, birds: roosters, turkeys, geese, ducks, ostriches, emu, fish).
      4.2.2. Breeding animals: male or female animals with outstanding performance and genetic features, designated for further breeding. To distinguish breeding animals (their above the average breeding value) we usually use prefix “breeding” (i.e. breeding sow, breeding mare, etc.).
   4.3. Hobby animals: Pets or companion animals are often home-dwelling animals, which are cared by humans and with which they cling as companions. Such can be e.g. dogs, cats, horses, some rodents, fishes, amphibians, reptiles and birds.
   4.4. Wild vertebrates: Vertebrates living in their natural habitat.
   4.5. Endangered and exotic animals: Severely endangered species are those species that have an exceptionally high risk of extinction in their natural habitat and are likely to become extinct in the wild without immediate intervention (Status iucn CR icon.svg). The Red List of the World

Conservation Union classifies such species and subspecies into the Critically Endangered category, abbreviated CR. This is the most severe threat status used in the Red List for the species still alive in their original habitat (excluding the Extinct and Wild Extinct statuses). According to the 2010 version of the Red List, 1,859 species of animals are on the verge of extinction.
4.6. Laboratory animals: animals which are bred for experimental purposes and are used for various tasks, such as drug testing, biological-, genetic- and biotechnological testing. Such animals are: mice, rats, cats, dogs, pigs, Drosophila and many other animal species.
5. Reproductive capacity: the ability of living subjects to reproduce themselves. The reproductive capacity of male vertebrates depends on the number of male gametes, i.e. sperm, and their physiological characteristics, primarily their motility and fertility.
6. Behavior: The set of reactions, responses, or movements that a subject performs in a given situation. Types of the behavior are innate or learned.
   6.1. Aggression: Aggression is an energetic, impulsive very often harmful form of social interaction, which can be:
      negative, when the aim of the behavior is to cause damage or harm or
      positive, when it is for purposeful pursuit, creation, self-realization (human).

From another point of view, the behavior, when partners are fighting for different resources (food, mating partner etc.) is also an aggressive behavior.

At the laboratory settings, aggressive behavior can be tested i.e. with social interaction test.
6.2. Anxiety: Restlessness, fear. A “standby” status related to an actual or perceived threat. Rodents are afraid of open spaces, or predator smells and try to avoid those. In humans, an event that is considered unpleasant, fear of public, or a possible recurrence of a previously experienced trauma can cause anxiety.
6.3. Obsessive-compulsive disorder (OCD) is a disorder in which people have recurring, unwanted thoughts, ideas or sensations (obsessions) that make them feel driven to do something repetitively (compulsions). In rodents OCD behavior can be tested with marble burying test.
7. Marble burying test: The Marble burying test is a test to measure the behavior of an animal model that is used to measure anxiety or obsessive-compulsive (OCD) behaviors: the test is based on the observation, that laboratory rodents harmful or harmless objects in the litter. For example, if rodents are placed in a cage containing marble balls, the animals will bury the balls in the litter over time. This behavior is related to anxiety or OCD. When rodents are injected with anxiolytics or drugs used to treat OCD, the amount of buried balls decreases.

### FIELD OF THE INVENTION

Decrease of fertility problems/disorders. Maintenance of normal, healthy fertility in male vertebrates and men. Restoration of normal sperm count and sperm quality, which has been destroyed due to aging, various environmental toxicants, malnutrition and/or excessive stress. Elevation of declining testosterone levels in andropause. Change the aggressive behavior of male breeding animals in a calm, peaceful direction. Reduction of anxiety. Reduction of obesity, increase of muscle mass in humans and vertebrates.

### The Biological Background of the Invention

The reproductive capacity of male vertebrates depends on the number of male gametes, i.e. sperms, and on their physiological characteristics, in particular their motility and fertilization capacity. Spermatogenesis taking place in the testis, in the seminiferous tubules, with the aid of Sertoli cells. The whole process takes for approx. 5-8 weeks. The activity of the spermatozoa is aided by secretions from the additional glands along the urinary tract. The amount of semen and the number of sperms in it varies depending on the species: mouse: 9.5 million pcs/ml; rats: 48 million 33 pcs/ml; porcine male: 154-500 ml, (100,000 pcs/ml); stallion: 30-200 ml, (100,000 pcs/ml); rooster: 0.3 to 0.8 ml (2 to 3.5 million/ml); turkey 0.3-0.5 ml ((3-6 million pcs/ml). Healthy young man: 1.5-2 ml, (33-36 million pcs/ml. In aging animals and men, the chances of a decrease in sperm count increase over time compared to the young population.

Research shows that the sperm count of men decreased by 40% in the last 50 years. Although no such drastic reduction has occurred among farm animals, the quality and quantity of semen from donor male animals used in artificial insemination is also important in animal husbandry. Increasing the sperm count and quality may reduce the number of sperm donors and thus the cost of animal maintenance. Maintaining and enhancing fertility is an equally important aspect in the breeding of pets. Increasing sperm count and thus the reproductive capacity is also essential in rescuing endangered species in order to achieve adequate offspring.

Testosterone levels (blood plasma/serum): The most important male sex hormone, testosterone, is synthesized by testicular (interstitial) Leydig cells. By acting on the Sertoli cells, testosterone helps to differentiate normal spermatozoa from spermatogonia and is involved in regulation of male-specific behaviors. Plasma levels of testosterone in laboratory rodents: mouse: 1-2 ng/ml; rat: 2-2.5 ng/ml; In other species: porcine male: 4.5-8 15 ng/ml, stallion: 1.9-2.1 ng/ml, healthy young male: 2.5-8.6 ng/ml, aging male: 1.9-7.3 ng/ml. Maintaining of healthy testosterone levels in an aging body is important not only for reproduction but also for cardiovascular and metabolic health.

Testosterone has an effect—through the central nervous system—on behavior. It is generally held that testosterone is hormone of dominance in both humans and animals. In species living in community, both pro-social (mutually supportive) and anti-social, aggressive testosteronedependent behaviors can be observed.

In human and veterinary medicine, various vitamin preparations (vitamins A, B, C, D, E), antioxidants (ferulic acid, Coenzyme Q, carnitine, green tea), micro- and macroelements (Fe, Zn, Se, Cu, Mn, Cr) are used to increase sperm count, viability and fertility. Of the herbs, crows foot (Tribulus terrestris), aswagandha (Withania somnifera) and fenugreek (Trigonella foenum-graecum) are used. Although some formulations contain several components listed, unfortunately such treatments are not specific and not effective enough.

In the course of the present invention, it has been found that compositions of Lactiplantibacillus plantarum SNI3 are effective in improving male fertility/reproductive performance.

### STATE OF THE ART

US patent US2021023147 (A1) elicitation describes a Lactobacillus strain which decreases high blood glucose levels and has antioxidant activity.

Canadian Publication CA3105723 (A1) for use of reutericycline or an analog thereof to reduce weight gain or induce weight loss.

The invention of WO2017223364 (A1) relates to the use of Lactobacillus reuteri bacteria isolated from canine saliva to modify the body weight of a subject.

The food supplement called Probio24 (Nutraceutics Ltd.) (https://probio24.nutraceutics.hu/) a commercially available probiotics complex of containing 11 different strains of probiotic bacteria: Lactobacillus acidophilus, Bifidobacterium lactis, Lactobacillus rhamnosus, Bifidobacterium breve, Lactobacillus bulgaricus, Lactobacillus plantarum, Lactobacillus casei and Lactobacillus lactis.

The capsules of the dietary supplement called Bio-Kult (https://bio-kult.hu/) (distributed by Vitaminkosár GmbH) contains 14 bacterial strains: (Lactobacillus paracasei, Lactobacillus plantarum, Lactobacillus rhamnosus, Bacillus subtilis, Bifidobacterium bifidum, Bifidobacterium breve, Bifidobacterium longum, Lactobacillus acidophilus, Lactobacillus lactis, Streptococcus thermophilus, Bifidobacterium infantis, Lactobacillus bulgaricus, Lactobacillus helveticus, and Lactobacillus salivarius).

Publications by Borre et al. and Codagnone et al. the microbiome is a symbiotic community of pro- and eukaryotic organisms, which have a significant effect on host physiology. (1,2).

Palleja et al. report that the microbiome is found on the body-environment interface, the number of species consisting the microbiome is estimated several thousands. The best studied part of the microbiome is the community which populates the gut (gut microbiome) (3).

According to the publications by Ghosh et al. and Saeedi et al., although Lactobacilli represent only a minor part of the gut microbiome, they have a significant beneficial effect on the host functions (4,5)

Publications by Poutahidis et al., Warda et al., Thomas et al. and Prete et al., reveal that among the probiotic bacteria, which confer beneficial effects for the host, the most prominent genus is Lactobacillus. Lactic acid bacteria have profound effect on the gut function by displacing harmful species. They stimulate immune functions, mucosal immunity, have anti-obesity actions and produce anti-inflammatory and anti-oxidant metabolites (6-9).

Publications by Poutahidis et al., Fadda et al., Sheikh et al., Varian et al. from the past few years, refer to that certain lactic acid bacteria might have beneficial effect on male reproductive functions (1013).

### The Recognition

During the development of our invention, results of our experiments reveal that our bacterial isolate (deposited as NCAIM (P) B110482) referred to as Lactiplantibacillus plantarum SNI3 has:
pro-reproductive (increase of sperm number and testosterone level) effect
psychobiotic (anxiolytic and anti-aggressive) effect□ and decreases body fat.

This new, complex and beneficial bodily and behavioral effects have been tested in laboratory rodent models.

### Description of a General Solution in Accordance with the Main Claim

Our invention covers our bacterial isolate Lactiplantibacillus plantarum SNI3 strain, which have been deposited as NCAIM (P) B110482, preparations containing Lactiplantibacillus plantarum SNI3 strain and application of the preparation on male vertebrate animals, breeding animals and men to change their behavior, lean body mass to favorable direction, to reduce their body fat, to increase reproductive capacity, sperm count and quality as well as to increase testosterone levels.

According to one preferred embodiment of the preparation in accordance with the invention, contains 1×10¹-10¹¹ CFU (colony-forming units), of SNI3 bacteria per milliliter or gram of food supplement, drinking water, drinking juice, feed, food, feed/food supplement.

The preparation preferably contains the living bacterium strain, the heat inactivated bacterium, bacterial lysate, any bacterial component and/or metabolite.

The invention even relates to the use of a preparation/composition comprising the SNI3 bacterial strain. The application of the composition is for improving the reproductive status of male vertebrates and human men, increasing sperm count, reducing aggression and anxiety.

The composition may also contain herbs, mannose or manno-oligosaccharides.

In an embodiment of the invention said composition is a liquid formulation or a solid formulation as a feed- or food supplement, wherein said solid formulation is selected from the group consisting of powders, tablets, chewing tablets, capsules, and said liquid formulation is selected from the group consisting of oral solutions, emulsions, syrups and milk products.

In a further embodiment of the invention said composition comprising the bacterium Lactiplantibacillus plantarum SNI3, prepared in the known manner and, if desired, pretreated with heat or ultrasound, will be prepared with carriers and excipients selected from those commonly used in pharmaceutical technology, into pharmaceutical preparation or food supplement.

Our experiments were performed between 2018-2021 on adult male C57Bl6 mice and Wistar rats. Animals were kept under standard laboratory conditions (temperature: 21±1° C.; humidity: 50% light dark cycle: 12 h dark and 12 h light) at the minimal disease (MD) level of the animal facility of the Institute of Experimental Medicine, Eötvös Loránd Research Network, Budapest, Hungary). Animals received standard rodent food ad libitum.

Lactiplantibacillus plantarum SNI3 stain was grown in a closed bottle in MRS (deMan, Rogosa, and Sharpe medium) for 48 h at 37° C., then bacterial cultures were centrifuged and bacteria resuspended in sterile drinking water. Animals received Lactiplantibacillus plantarum SNI3 suspension for four weeks and the bottles containing the bacterial solution were changed daily. The concentration of the Lactiplantibacillus plantarum SNI3 was 1-5×106 CFU/ml. Control animals received sterile water to drink.

In cases, when animals drunk cell free supernatant of Lactiplantibacillus plantarum SNI3 cultures for 4 weeks, control animals received MRS medium to drink.

In other cases, Lactiplantibacillus plantarum SNI3 cells were suspended in physiological saline and then exposed to 80° C. heat for 1 h. Heat treated bacteria were mixed to sterile drinking water and were given to animals as drinking fluid for 4 weeks.

In a yet other case, Lactiplantibacillus plantarum SNI3 cells were suspended in physiological saline and sonicated. Then the cell lysate have been passed through 0.2 μm filter and was given to animals for drinking for 4 weeks.

The body weight of the animals was measured weekly, the body composition was analyzed before the start of the treatment and at the end of the experiment by EchoMRI™.

At the end of the experiment a social interaction test and marble burying test have been performed. The animals have been decapitated, trunk blood collected, centrifuged and testosterone concentration has been measured from the serum using ELISA. Sperm were isolated from the cauda epididymis and counted in Bürker chamber. The weight of dissected thymus, adrenals, spleen and testes was measured. The left lobe of the liver, the colon and one testis were frozen in liquid N2, The colon content samples were frozen in dry-ice. The other testis was fixed in formalin, embedded in paraffin to prepare histological sections for hematoxylin-eosin staining.

The use of the active ingredient is illustrated by the following examples and the figures relating to the examples.

### LIST OF FIGURES

1. Effect of living Lactiplantibacillus plantarum SNI3 treatment on the percentage of body fat in male mice.
2. Effect of Lactiplantibacillus plantarum SNI3 treatment on the elements of aggressive behavior in male mice.
3. The results of marble burying test.
4. The relative testis weight in the treated vs control groups.
5. Histology of testes obtained from Lactiplantibacillus plantarum SNI3-treated and control mice.
6. Area of the seminiferous tubules in the testes of Lactiplantibacillus plantarum SNI3-treated and control mice.
7. Thickening of the germinal epithelium in the testis of Lactiplantibacillus plantarum SNI3 treated mice, compared to controls.
8. The number of spermatozoa isolated from the epididymis.
9. Changes in serum testosterone levels.
10. The effect of different forms of Lactiplantibacillus plantarum SNI3 on the testis weight of the mice.
11. The effect of different forms of Lactiplantibacillus plantarum SNI3 on the sperm count in mice.
12. The number of sperms isolated from epididymis of rats treated with Lactiplantibacillus plantarum SNI3 compared to controls.
13. Effect of different (prebiotic) carbohydrates on the cell number of Lactiplantibacillus plantarum SNI3.

On FIG. 1, the effect of treatment with the living Lactiplantibacillus plantarum SNI3 on the body fat is seen. It has been revealed that the relative body fat content decreased in response to Lactiplantibacillus plantarum SNI3 treatment: Data obtained by EchoMRI, Mean+SEM values, non-paired t-test, p=0.014.

On FIG. 2, the effect of Lactiplantibacillus plantarum SNI3 on the frequency of aggressive behavioral elements (threat, attack, bite) in a social interaction test is seen. Mean+SEM values, non-paired t-test, p=0.05. It is well seen that the frequency of aggressive behavioral elements are closed to zero, compared to the behavior of control animal's “3” value.

On FIG. 3, the results of marble burying test are seen. This test measures anxiety. Mice bury strange objects such as marble balls placed in their cages, the number of balls buried isproportional to the degree of anxiety. The figure shows the number of balls buried by control (tap water) and Lactiplantibacillus plantarum SNI3-treated mice. Average+SEM. Unpaired t-test, p=0.0011.

FIG. 4 shows the effect of living Lactiplantibacillus plantarum SNI3 on relative testis weight (mg testis weight/g body weight). The bacterium solution was given to the mice through drinking water for 4 weeks. Average+SEM. Unpaired t-test, p=0.0011.

On FIG. 5 photomicrographs of hematoxylin-eosin stained testis sections are found. Compared to mice drinking sterile tap water, on the sections from Lactiplantibacillus plantarum SNI3-treated mice the enlargement of the areas of seminiferous tubules, germinative epithelium and interstitial cells can be observed.

FIG. 6 shows quantitative changes of seminiferous tubules areas. In mice, drunk tap water for 4 weeks, the area of 16500 μm² increased to 17500 μm² in mice treated with Lactiplantibacillus plantarum SNI3, meaning 6% increase of the area. Average+SEM. Unpaired t-test, p<0.05.

On FIG. 7 the thickening of the germinative epithelium is seen in the animals drunk Lactiplantibacillus plantarum SNI3 solution for 4 weeks compared to that of mice drunk tap water. Average+SEM. Unpaired t-test, p=0,0002.

On FIG. 8 it is well recognized that the number of spermatozoa isolated from the epididymis is increased in mice that have been treated with Lactiplantibacillus plantarum SNI3 solution compared to the controls. The sperm count increased 3× in the treated group, compared to those drunk tap water.

FIG. 9 shows the changes in serum testosterone levels in control (drinking tap water) and in mice, treated with Lactiplantibacillus plantarum SNI3 composition for 4 weeks. There was a 8× increase of testosterone level in the treated group. Data obtained by ELISA, Average+SEM. Unpaired t-test, p=0.003.

On FIG. 10 the effect of different forms* of Lactiplantibacillus plantarum SNI3 on the mouse testis weight is seen. (*live bacterium, cell free supernatant of the bacterium culture, bacterium lysate and heat inactivated bacterium.)

On FIG. 11, one can clearly see the sperm numbers (isolated from the epididymis) of mice drinking:
tap water (control)
live Lactiplantibacillus plantarum SNI3,
cell free supernatant of the SNI3 bacterium culture,
SNI3 bacterium lysate,
and heat inactivated SNI3 bacterium.

Average+SEM. Unpaired t-test, ***p=0.0024; *p=0.0154.

The highest sperm count was found in the group of mice, which have been treated with live Lactiplantibacillus plantarum SNI3, 2.6 fold change, compared to the control.

FIG. 12 shows changes in sperm count in Wistar rats given Lactiplantibacillus plantarum SNI3 composition compared to controls. It is concluded that the sperm count of the treated group is 1.3×higher than that of the untreated control group. Average+SEM. Unpaired ttest, p=0.0402.

On FIG. 13, the effect of various carbohydrates (added in 2% to the culture medium) on the growth of Lactiplantibacillus plantarum SNI3 culture (37° ° C., 48 h) as measured by the optical density. Carbohydrate free MRS medium: MRS-G; G: glucose, M: mannose; MO: mannooligosaccharides. It is well recognized from the figure that addition of mannose and mannooligosaccharides are the best for the growth of Lactiplantibacillus plantarum SNI3.

### EXAMPLES

### Example 1. Effect of Lactiplantibacillus plantarum SNI3 Composition on Male C57Bl6 Mice

To study the potential therapeutic effect of Lactiplantibacillus plantarum SNI3 bacterium, adult male mice were randomly assigned into two groups:

One group of mice (control) received filter-sterilized tap water the other group received live bacterial suspension of Lactiplantibacillus plantarum SNI3 suspended in filter-sterilized tap water, in a dose of 1-5×10⁶ CFU/ml for 4 weeks.
1a. mice received filtered tap water to drink for 4 weeks
1b. mice received Lactiplantibacillus plantarum SNI3 composition for 4 weeks

The effect of the bacterial composition on the body composition: at the end of the 4 week treatment period, the relative fat mass of Lactiplantibacillus plantarum SNI3-treated mice decreased from 8% to 5%. Data obtained by Echo-MRI. Mean+SEM values, non-paired t-test, p=0,014. FIG. 1 shows body fat content of mice, relative to the body weight: a.) in mice drunk filtered tap water or b.) in mice drunk Lactiplantibacillus plantarum SNI3 composition.

### Example 2

The procedure was the same as in Example 1, except that in this case, aggressive behavior was studied in a social interaction test. The effect of Lactiplantibacillus plantarum SNI3 composition on the social (aggressive) behavior is seen on FIG. 2. In this figure we show that the frequency of aggressive behavioral elements are approx.3, while the aggressive behavior of mice treated for 4 weeks with Lactiplantibacillus plantarum SNI3 composition decreased almost to zero. (Mean+SEM values, non-paired t-test, p=0.05).

### Example 3. Effect of Lactiplantibacillus plantarum SNI3 Composition on the Anxiety

The procedure was the same as in Example 1, except that in this case Marble burying test was performed in control and treated mice. This test measures anxiety and obsessive-compulsive behavior. The less marble balls are buried, the less anxious are the animals. This test revealed that mice treated with Lactiplantibacillus plantarum SNI3 are significantly less anxious compared to those treated with tap water only (FIG. 3).

On FIG. 3, we show that 4 weeks treatment of mice with Lactiplantibacillus plantarum SNI3 composition decreased the anxiety of the animals. The result of the Marble burying test: the number of buried balls was less in those animals that received Lactiplantibacillus plantarum SNI3 composition compared to those that drunk tap water. On FIG. 3, the number of balls buried by control (drinking tap water) and by SNI3-treated mice is shown. (Mean+SEM values, non-paired t-test, p=0.0011.)

### Example 4. Effect of Lactiplantibacillus plantarum SNI3 on the Mouse Testis Weight

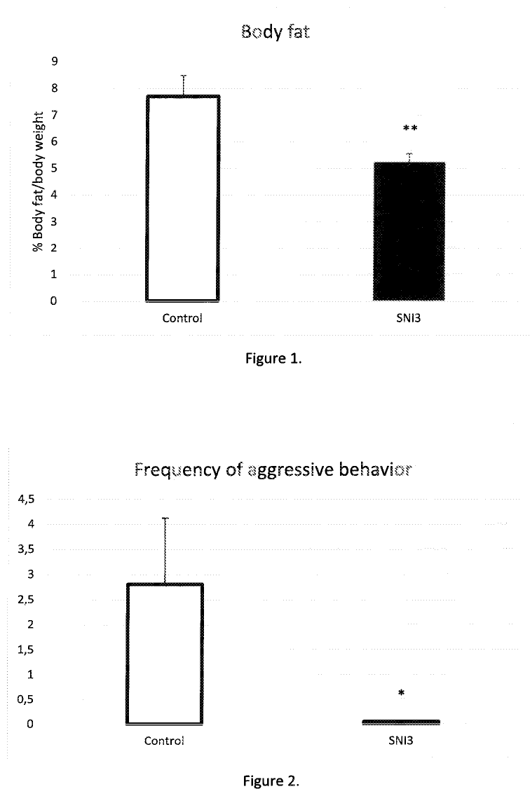

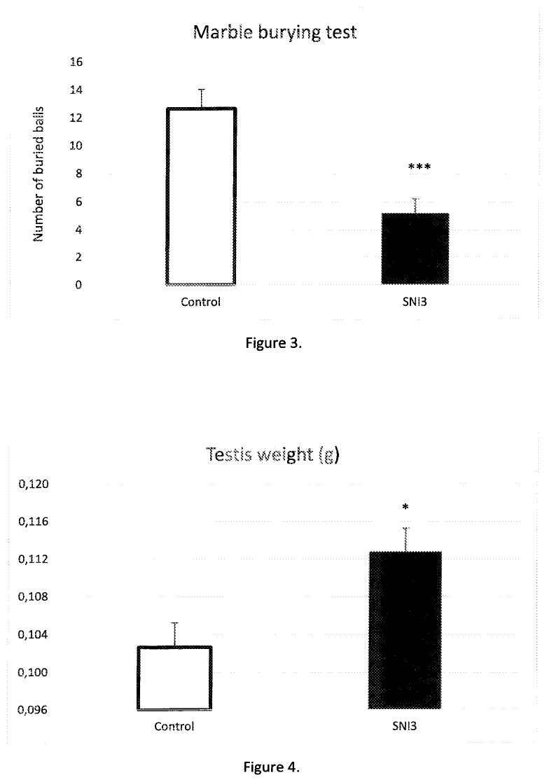

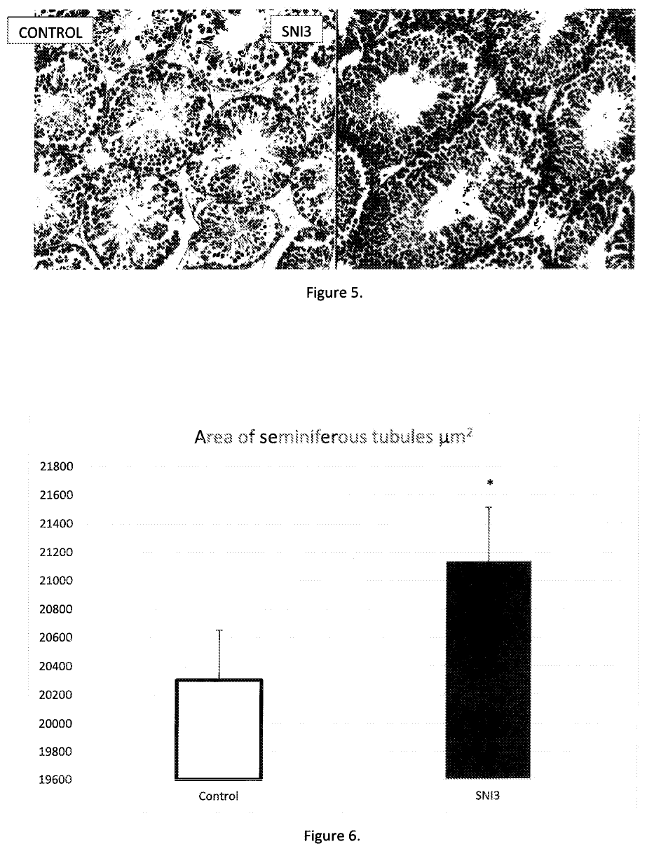

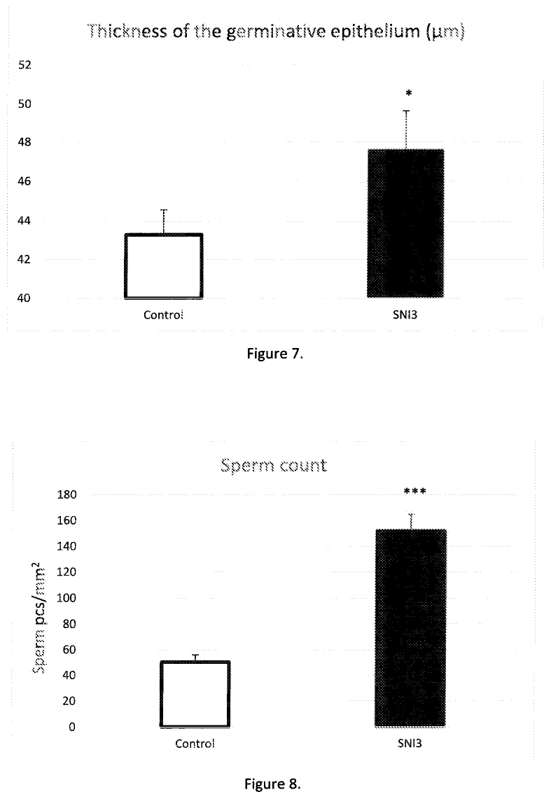

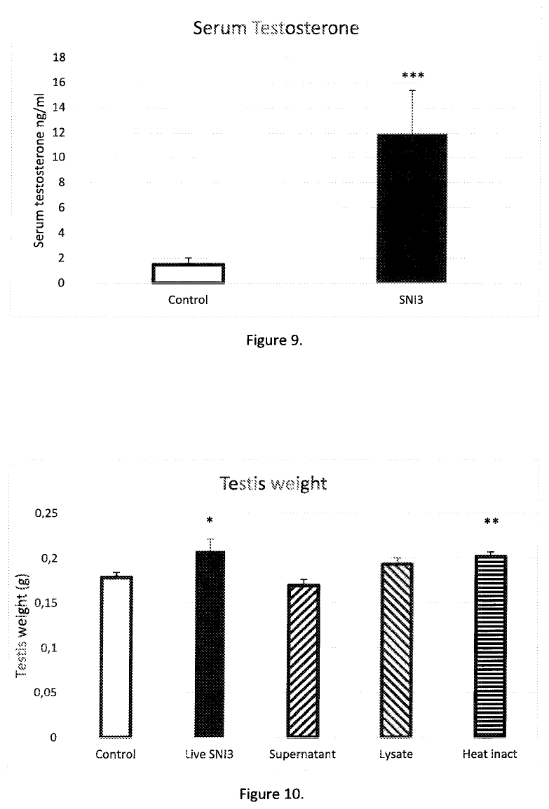

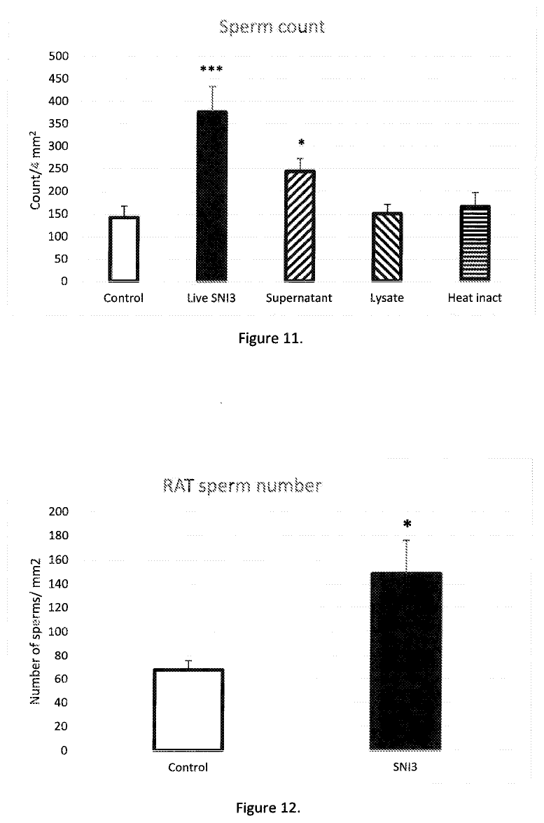

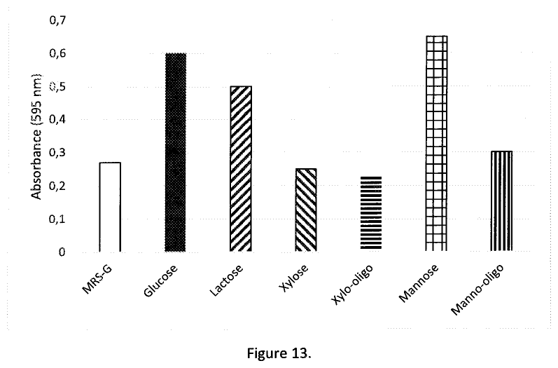

The procedure was the same as in Example 1. At the end of the experiment, mice were decapitated, the testes were removed and weighted. It has been shown that the absolute and relative weight (normalized to body weight) of the testis was increased in Lactiplantibacillus plantarum SNI3-treated mice compared to controls. The results of the testis measurement is shown on FIG. 4 as the testis weight of Lactiplantibacillus plantarum SNI3-treated mice was significantly higher than those of control animals (drinking tap water). The testis weight of Lactiplantibacillus plantarum SNI3-treated mice was 1.11 times higher than that of the controls. On FIG. 4, we show the increase of normalized testis weight after the administration of the composition. The effect of drinking for 4 weeks of live Lactiplantibacillus plantarum SNI3 composition on the testis weight (relative to body weight: testis weight in mg/body weight in g). Mean+SEM values, non-paired t-test, p=0.0114. Along this line, we have examined the histology of the testis. On FIG. 5, we demonstrate the histology of the testis, on FIG. 6 the changes of the seminiferous tubules' area and on FIG. 7, the thickening of the germinative epithelium.

### Example 5. Effect of Lactiplantibacillus plantarum SNI3 Composition on the Sperm Count

The procedure was the same as in Example 1. At the end of the experiment, we have counted the number of spermatozoa, which have been isolated from the cauda epididymis. The sperm number was 3 times higher in mice, which consumed Lactiplantibacillus plantarum SNI3 composition compared to the controls. On FIG. 8, we demonstrate the number of sperms from Lactiplantibacillus plantarum SNI3-treated mice and from tap-water drinking control animals. Mean sperm number/μm2+SEM, non-paired t-test, p<0.001.

### Example 6. The Effect of Lactiplantibacillus plantarum SNI3 Composition on the Serum Testosterone Level in Mice

The procedure was the same as in Example 1. At the end of the experiment we have measured serum testosterone concentration using ELISA. According to the results, the testosterone concentration in mice treated with Lactiplantibacillus plantarum SNI3 composition was 11.9±3.46 ng/ml, compared to 1.46±0.54 ng/ml of the tap-water controls (8.15 fold increase).

### Example 7

Comparison of the effects of live Lactiplantibacillus plantarum SNI3, cell free supernatant of the bacterial medium, heat-inactivated and bacterial lysate.

To investigate the active component of the Lactiplantibacillus plantarum SNI3, adult male mice were divided into the following 5 groups: 1: control mice drinking sterile filtered tap water; 2: mice drinking Lactiplantibacillus plantarum SNI3 composition, containing 1-5×10⁶ CFU/ml live bacteria mixed in sterile filtered tap water; 3: mice drinking a cell free supernatant of 48 h Lactiplantibacillus plantarum SNI3 culture medium; 4. mice drinking lysed suspension of 1-5×10⁶ CFU/ml Lactiplantibacillus plantarum SNI3. (The lysate was prepared as follows: the bacterial suspension was digested with lizozyme (4 h, 37° C.) sonicated 25×1 min, 20 kHz and filtered through a 20 μm filter.) 5. mice drinking heat inactivated (80° ° C., 1 h) form of Lactiplantibacillus plantarum SNI3 composition, containing 1-5×10⁶ CFU/ml. Animals were treated with these bacterial forms for 4 weeks. FIG. 10 demonstrates the effects of various bacterial preparations on the testis weight. The live Lactiplantibacillus plantarum SNI3 displayed highest testis growth promoting effect, but the heating activated form also had a significant effect.

FIG. 11 demonstrates the effects of various bacterial preparations on the sperm number. At the end of the experiment, spermatozoa were prepared from the epididymis and counted in a Bürker chamber. As seen on FIG. 11, the highest elevation of sperm number was seen in the group, which has been treated with live bacteria, but a significant elevation was also found in the group, which has been treated with cell free supernatant of Lactiplantibacillus plantarum SNI3 culture.

### Example 8. Effect of Lactiplantibacillus plantarum SNI3 Composition on the Sperm Number of Rats

In this study, adult male Wistar rats were divided into two groups: one group of animals received sterile filtered water to drink, the other group of rats drunk Lactiplantibacillus plantarum SNI3 composition mixed into sterile tap water, the dose was 1-5×10⁶ CFU/ml. Both treatments were 4 weeks. At the end of the experiment after autopsy, spermatozoa were prepared from the epididymis and counted in Bürker chamber. FIG. 12 shows the results of the experiment. FIG. 12 shows that live Lactiplantibacillus plantarum SNI3 composition resulted in 30% increase of sperm number in rats.

### Example 9. Effect of Prebiotics on the Growth of Lactiplantibacillus plantarum SNI3

The positive actions of Lactiplantibacillus plantarum SNI3 can be further supported by prebiotics. Prebiotics are compounds, which promote the growth or action the beneficial bacteria, by providing special bacterial food. The following prebiotics have been tested: lactose, xylose, mannose, xylooligosaccharide and mannooligosaccharide. Their effect was compared to glucose, which is the most preferred bacterial fuel. Bacterial growth was followed by measuring optical density of bacterial cultures at 595 nm using Filtermax Multimode Reader, in the presence of different carbohydrates. Data was fitted on Baranyi model and growth parameters (growth speed, lag phase, and maximal cell density) were determined. Carbohydrate free MRS medium was used as negative control, while the test materials were added in 0.2% concentration. FIG. 13 shows the effects of various carbohydrates on the cell density of Lactiplantibacillus plantarum SNI3. As seen on FIG. 13, mannose and mannooligosaccharides are the best to support the growth of Lactiplantibacillus plantarum SNI3. Aloe vera is the best source of these carbohydrates.

### Description of Advantages Obtained by Using the Solution According to the Invention

Lactiplantibacillus plantarum strain SNI3 (deposited as NCAIM (P) B 001482) microbial-based preparations, in a manner not previously known, show simultaneously the following positive physiological and mental effects:
decrease in body fat percentage (more than 30%)
anxiolytic activity;
increase in relative weight of testis and area of seminiferous ducts, thickening of germinal epithelium (<10%)
increase in sperm count (<1.5-fold);
increase in testosterone levels (5-8 times)
increase in testosterone levels is not associated with otherwise suspected aggressive behavior, even the value of aggression is practically close to zero.

### REFERENCES

[1] Borre Y E, Moloney R D, Clarke G, Dinan T G, Cryan J F. The impact of microbiota on brain and behavior: 5 mechanisms & therapeutic potential. Adv Exp Med Biol. 2014; 817:373-403.
[2] Codagnone M G, Spichak S, O'Mahony S M, O'Leary O F, Clarke G, Stanton C, et al. Programming Bugs: Microbiota 7 and the Developmental Origins of Brain Health and Disease. Biol Psychiatry. 2019; 85(2):150-63.
[3] Palleja A, Mikkelsen K H, Forslund S K, Kashani A, Allin K H, Nielsen T, et al. Recovery of gut microbiota of healthy 9 adults following antibiotic exposure. Nat Microbiol. 2018; 3(11): 1255-65.
[4] Ghosh T S, Arnoux J, O'Toole P W. Metagenomic analysis reveals distinct patterns of gut lactobacillus prevalence, 11 abundance, and geographical variation in health and disease. Gut Microbes. 2020; 12(1):1-19.
[5] Saeedi B J, Liu K H, Owens J A, Hunter-Chang S, Camacho M C, Eboka R U, et al. Gut-Resident Lactobacilli Activate 13 Hepatic Nrf2 and Protect Against Oxidative Liver Injury. Cell Metab. 2020; 31(5):956-68 e5.
[6] Poutahidis T, Kearney S M, Levkovich T, Qi P, Varian B J, Lakritz J R, et al. Microbial symbionts accelerate wound 15 healing via the neuropeptide hormone oxytocin. PLOS One. 2013; 8(10):e78898.
[7] Warda A K, Rea K, Fitzgerald P, Hueston C, Gonzalez-Tortuero E, Dinan T G, et al. Heat-killed lactobacilli alter 17 both microbiota composition and behaviour. Behav Brain Res. 2019; 362:21323.
[8] Thomas C M, Hong T, van Pijkeren J P, Hemarajata P, Trinh D V, Hu W, et al. Histamine derived from probiotic 19 Lactobacillus reuteri suppresses TNF via modulation of PKA and ERK signaling. PLOS One. 2012; 7(2):e31951.
[9] Prete R, Long S L, Gallardo A L, Gahan C G, Corsetti A, Joyce S A. Beneficial bile acid metabolism from Lactobacillus 21 plantarum of food origin. Sci Rep. 2020; 10(1):1165.
[10] Poutahidis T, Springer A, Levkovich T, Qi P, Varian B J, Lakritz J R, et al. Probiotic microbes sustain youthful serum 23 testosterone levels and testicular size in aging mice. PLoS One. 2014; 9(1):e84877.
[11] Fadda L M, Alhusaini A M, Al-Qahtani Q H, Ali H M, Hasan I H. Role of alpha-tocopherol and Lactobacillus 25 plantarum in the alleviation of mercuric chloride-induced testicular atrophy in rat's model: Implication of molecular 26 mechanisms. J Biochem Mol Toxicol. 2020; 34(6):e22481.
[12] Sheikh Hosseini S, Gol A, Khaleghi M. The effects of the Lactobacillus acidophilus ATCC 4356 on the oxidative 28 stress of reproductive system in diabetic male rats. Int J Reprod Biomed. 2019; 17(7):493-502.
[13] Varian B J, Poutahidis T, DiBenedictis B T, Levkovich T, Ibrahim Y, Didyk E, et al. Microbial lysate upregulates host 30 oxytocin. Brain Behav Immun. 2017; 61:36-49

1. Lactiplantibacillus plantarum SNI3 bacterium strain, deposited as NCAIM (P) B 001482.

2. Composition, containing Lactiplantibacillus plantarum SNI3 bacterium strain of claim 1, wherein the 1 gram or 1 milliliter of food supplement, drinking water, dinking juice, nutrient, forage, and/or food, feed supplement contains the bacterial strain of claim 1 in concentration of 101-1011 CFU/ml and 101-1011 CFU/g respectively.

3. The composition of claim 2, wherein the preparation contains live Lactiplantibacillus plantarum SNI3 bacterium strain, heat killed bacterium, bacterium lysate, any component of the bacterium and/or its metabolite.

4. A method to increase the reproductive condition by 10-25%, to increase the sperm count at least 10-25%, to increase the serum testosterone level 1.5-5 fold, and/or to decrease aggression and anxiety at least 10-25% on male vertebrate animals and men, comprising administering to one or more male vertebrate animals and/or one or more men an effective amount of a preparation comprising the composition of claim 2.

5. Preparation according to claim 2, further comprising herbs, mannose or manno-oligosaccharides.

6. A method to favorably alter the behavior of breeding animals and human males comprising administering to male vertebrate animals, breeding animals and/or human males an effective amount of the composition according claim 2:
to increase reproductive condition
to increase sperm count,
to increase testosterone serum level,
to increase lean muscle mass and decrease fat mass, and/or
to decrease aggressivity and anxiety.

7. Powder, solution, syrup, dairy product, capsule, tablet or chewable tablet, comprising the composition of claim 2.
